(19) United States
(12) Patent Application Publication
(10) Pub. No.: US 2021/0333231 A1
(43) Pub. Date: Oct. 28, 2021

(54) pH MEASUREMENT OF AN AQUEOUS SAMPLE

(71) Applicants: Hach Lange GmbH, Berlin (DE); University of Warwick, Coventry (GB)

(72) Inventors: Zoë Duncan, Daventry (GB); Mark Edward Newton, Kenilworth (GB)

(21) Appl. No.: 16/860,715

(22) Filed: Apr. 28, 2020

(51) Int. Cl. G01N 27/30; G01N 33/18

(52) U.S. Cl. CPC G01N 27/302; G01N 27/308; G01N 33/1886

(57) ABSTRACT

An embodiment provides a device for measuring pH in an aqueous sample, including: a primary electrode comprising a boron doped diamond-based electrode and a primary carbon region comprising a pH sensitive sp2 carbon region, wherein the primary carbon region comprises a portion of the surface area of a face of the primary electrode; a secondary electrode comprising a boron doped diamond-based electrode and a secondary carbon region, wherein the secondary carbon region comprises a portion of the surface area of a face of the secondary electrode, the portion of the surface area of a face of the secondary electrode being less than the portion of the surface area of a face of the primary electrode; at least one reference electrode; at least one auxiliary electrode; and a memory storing instructions executable by a processor to identify a pH of an aqueous sample by measuring an electrical potential between the at least one reference electrode and at least one of: the primary electrode and the secondary electrode. Other aspects are described and claimed.

### FIELD

This application relates generally to pH measurement of an aqueous sample, and, more particularly, to pH measurement using electrodes with a carbon region.

### BACKGROUND

Ensuring water quality is critical to the health and well-being of humans, animals, and plants, which are reliant on water for survival. One parameter of water that may be measured is the pH. The measurement of pH of an aqueous sample is critical in a number of industries such as pharmaceuticals, biomedical, water supply, and other manufacturing fields. Measurement of pH may allow for proper treatment of water or ensuring proper water quality, and allows for identifying the overall quality of the water. One method to measure pH in an aqueous sample includes the use of electrodes which require constant maintenance and calibration of the pH measurement system.

### BRIEF SUMMARY

In summary, one embodiment provides a device for measuring pH in an aqueous sample, comprising: a primary electrode comprising a boron doped diamond-based electrode and a primary carbon region comprising a pH sensitive sp2 carbon region, wherein the primary carbon region comprises a portion of the surface area of a face of the primary electrode; a secondary electrode comprising a boron doped diamond-based electrode and a secondary carbon region, wherein the secondary carbon region comprises a portion of the surface area of a face of the secondary electrode, the portion of the surface area of a face of the secondary electrode being less than the portion of the surface area of a face of the primary electrode; at least one reference electrode; at least one auxiliary electrode; and a memory storing instructions executable by a processor to identify a pH of an aqueous sample by measuring an electrical potential between the at least one reference electrode and at least one of: the primary electrode and the secondary electrode.

Another embodiment provides a method for measuring pH in an aqueous sample with a carbon region electrode, comprising: introducing an aqueous sample into a measurement device, wherein the measurement device comprises: a primary electrode comprising a boron doped diamond-based electrode and a primary carbon region with a primary carbon region surface area, wherein the primary carbon region comprises a pH sensitive sp2 carbon region; a secondary electrode comprising a boron doped diamond-based electrode and a secondary carbon region with a secondary carbon region surface area less than the primary carbon region surface area; at least one reference electrode; at least one auxiliary electrode; and identifying a pH of an aqueous sample by measuring an electrical potential between the at least one reference electrode and at least one of: the primary electrode and the secondary electrode.

A further embodiment provides a system for measuring pH in an aqueous sample, comprising: a storage device having code stored therewith, the code being executable by the processor and comprising: code that introduces an aqueous sample into a measurement device, wherein the measurement device comprises: a primary electrode comprising a boron doped diamond-based electrode and a primary carbon region with a primary carbon region surface area, wherein the primary carbon region comprises a pH sensitive sp2 carbon region; a secondary electrode comprising a boron doped diamond-based electrode and a secondary carbon region with a secondary carbon region surface area less than the primary carbon region surface area; at least one reference electrode; at least one auxiliary electrode; and code that identifies a pH of an aqueous sample by measuring an electrical potential between the at least one reference electrode and at least one of: the primary electrode and the secondary electrode.

The foregoing is a summary and thus may contain simplifications, generalizations, and omissions of detail; consequently, those skilled in the art will appreciate that the summary is illustrative only and is not intended to be in any way limiting.

For a better understanding of the embodiments, together with other and further features and advantages thereof, reference is made to the following description, taken in conjunction with the accompanying drawings. The scope of the invention will be pointed out in the appended claims.

### BRIEF DESCRIPTION OF THE SEVERAL VIEWS OF THE DRAWINGS

FIG. 1 illustrates a schematic diagram of a measurement electrode for measuring pH in an aqueous sample in an example embodiment.

FIG. 2 illustrates a flow diagram of measuring pH in an aqueous sample.

FIG. 3 illustrates an example of a measurement electrode for measuring pH in an aqueous sample in an example embodiment.

FIG. 4 illustrates an example of computer circuitry.

### DETAILED DESCRIPTION

It will be readily understood that the components of the embodiments, as generally described and illustrated in the figures herein, may be arranged and designed in a wide variety of different configurations in addition to the described example embodiments. Thus, the following more detailed description of the example embodiments, as represented in the figures, is not intended to limit the scope of the embodiments, as claimed, but is merely representative of example embodiments.

Reference throughout this specification to “one embodiment” or “an embodiment” (or the like) means that a particular feature, structure, or characteristic described in connection with the embodiment is included in at least one embodiment. Thus, appearances of the phrases “in one embodiment” or “in an embodiment” or the like in various places throughout this specification are not necessarily all referring to the same embodiment.

Furthermore, the described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well-known structures, materials, or operations are not shown or described in detail. The following description is intended only by way of example, and simply illustrates certain example embodiments.

The measurement of the pH of water or other aqueous solutions or samples is common and allows for determination of the quality or other characteristics of the aqueous solution. Conventional pH electrodes for measurement of pH may be constructed using fragile, thin glass. This glass breaks easily leading to higher replacement and maintenance costs. The possible breakage of glass pH electrodes may also limit their use in food and beverage applications. Conventional pH electrodes also may have “alkali errors.” These errors arise from interfering ions such as sodium and potassium affecting the pH response at high pH values. A commercial need exists for a robust pH measurement electrode that requires less maintenance while maintaining measurement of pH in sample containing heavy metals or a low conductivity sample.

Some methods to measure pH use an electrochemical signal in an aqueous sample. Aqueous samples may be from wastewater, industrial fluids, natural bodies of water, or the like. The aqueous sample may contain electrochemically active interferences such as heavy metals. Heavy metals may have their own electrochemical signal. Thus, measuring pH in an aqueous sample may be affected by a presence of heavy metals in the aqueous sample. Examples of heavy metals may include copper, manganese, lead, iron or the like.

Another method of measuring pH of an aqueous sample uses a laser machined boron-doped diamond (BDD) material to create a sensor capable of measuring pH. Laser machining of the BDD creates sp2 carbon material integrated into the BDD surface that contains pH sensitive quinone-like structures. These laser machined areas or pits may be in an array pattern. In other words, the laser machined areas may be a series of spots across a surface. The machined area may be determined by the size of a laser beam, the resolution of the beam, and may or may not cover an entire electrode surface.

A BDD electrode may give greater accuracy, especially improved accuracy in samples containing electrochemically active interferences. For example, a water sample may contain heavy metal or metals, and may require an accurate pH measurement. A traditional pH electrode system may measure both the electrochemical pH signal and the electrochemical interference signal by the of the heavy metal at the same time. The result may be an incorrect pH measurement. This may be caused, but is not limited to, signal convolution. BDD electrodes may measure pH in all pH ranges including am environmentally relevant range of pH 4 to 11. What is needed is an accurate system and method to measure pH in an aqueous sample containing one or more heavy metals. The system and method may measure an electrochemical signal from a heavy metal interference. The interference may be subtracted from a total signal to determine an accurate pH measurement.

Accordingly, the systems and methods as described herein may determine a pH by identifying an electrical potential of an aqueous sample. The system may measure at least two electrical potentials. The system may use two or more electrodes to measure a different electrochemical signal for each electrode. For example, a primary electrode may be a pH sensitive electrode. A secondary electrode may be sensitive to an interference electrochemical signal. A secondary electrode may be a BDD electrode. The secondary electrode may have no sp2 carbon region. In other words, zero surface area of the secondary electrode may be a laser machined sp2 carbon region. The interference electrochemical signal may be from a heavy metal component in the aqueous sample.

The primary electrode may have a carbon region and a pH sensitive carbon region. For example, the primary electrode may be a BDD electrode with a plurality of sp2 carbon regions. The pH sensitive carbon region may be laser machined. The carbon region may comprise sp2 carbon materials that can include diamond-like materials doped with elements like boron (BDD). The pH sensitive carbon region may be an sp2 carbon region that is included on a boron doped diamond-based pH electrode. Being included may mean that the sp2 carbon region is introduced into, integrated into, contained within, laser micro machined into, or otherwise integrated into the boron doped diamond electrode. In other words, while the sp2 carbon region and the boron doped diamond are integrated into the same electrode, they are chemically different regions of the electrode. The carbon region may have oxidized carbon structures. The oxidized carbon structure may have quinone or quinone-like groups. The method and system may also comprise at least one reference electrode and at least one auxiliary electrode. In an embodiment, an applied potential protocol may be applied to at least a primary measurement electrode. An electrical potential between the at least one measurement electrode may be measured. In an embodiment, the electrical potential may correspond to a pH measurement of an aqueous sample. The at least one measurement electrode, at least one reference electrode, and at least one auxiliary electrode may be operatively coupled to circuitry to measure, analyze, and store pH measurements of a sample.

The use of BDD serves as a better electrode material than other carbon-based or metallic materials (e.g., silver, gold, mercury, nickel, etc.) because these materials may be more electrocatalytically active, and may generate interfering signals and contributing to the errors in the measurement of pH.

The illustrated example embodiments will be best understood by reference to the figures. The following description is intended only by way of example, and simply illustrates certain example embodiments.

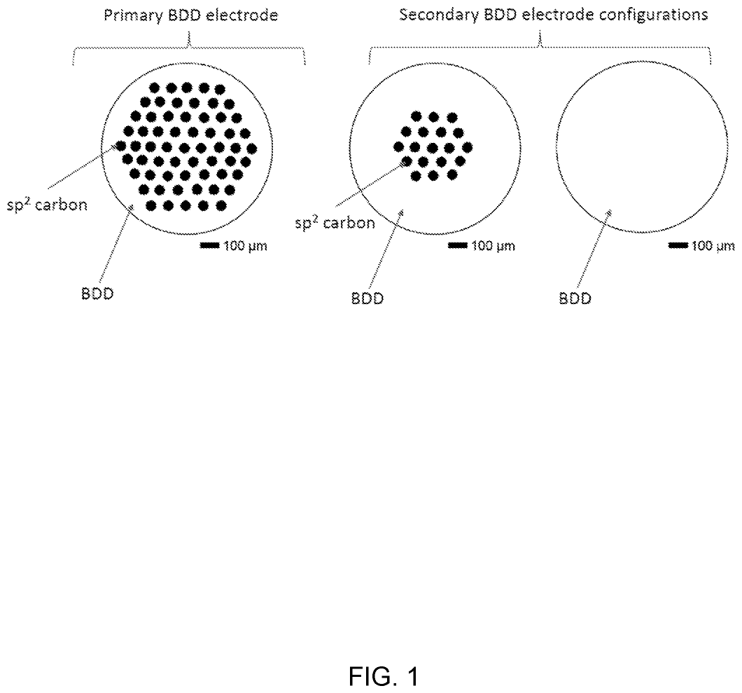

Referring now to FIG. 1, an embodiment may measure a characteristic of an aqueous solution using at least one measurement electrode. In an embodiment, the characteristic may be a pH measurement. The system may have at least one reference electrode. The system may have at least one auxiliary electrode. In an embodiment, the system may comprise a primary electrode. The primary electrode may be a BDD electrode with at least one sp2 carbon region. In an embodiment, the method and system may comprise a secondary electrode. The secondary electrode may be a BDD electrode with no sp2 region or an sp2 region with a lesser area than the sp2 area of a primary electrode. The primary electrode may be pH sensitive. The secondary electrode may be sensitive to an interferent. The secondary electrode may be pH sensitive, but less so than the primary. In an embodiment, the interferent may be subtracted from the pH signal for an accurate pH measurement. In an embodiment, the method and system may be a 3-electrode system. For example, the system may measure using an auxiliary, a reference, and either a primary or secondary electrode.

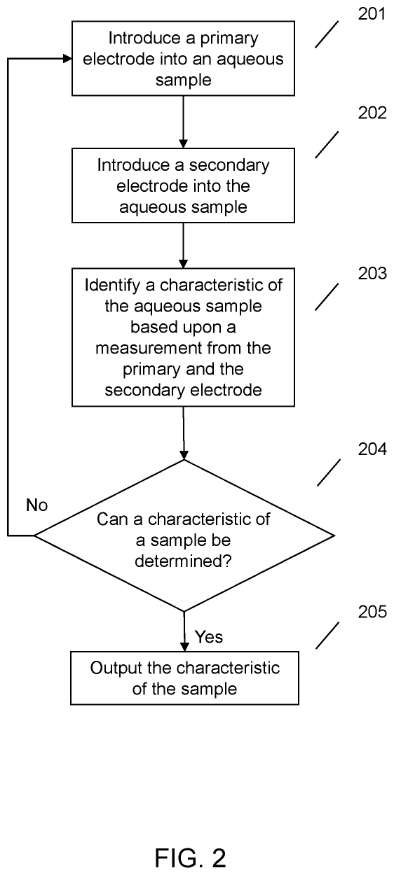

Referring to FIG. 2, an embodiment method for measuring a pH of an aqueous sample is illustrated. At 201 in an embodiment, a primary electrode may be introduced into an aqueous sample. A primary electrode may be referred to as a primary measurement electrode or a primary BDD electrode (see FIG. 1). In an embodiment, a primary electrode may be used to measure a pH of an aqueous sample. In an embodiment, a primary electrode may comprise a pH sensitive carbon region. In other words, a primary electrode may have a region that has been produced by laser machining for example. The pH sensitive region may be of a BDD material which may comprise sp2 carbon material. In other words, a primary electrode may be of a BDD material with at least one area of laser machined sp2 carbon area. In an embodiment, the sp2 carbon regions may be a plurality of laser machined areas. In an embodiment, the sp2 carbon may have a surface area. The sp2 carbon or carbon surface area may be a total surface area of all sp2 regions upon an electrode face.

The system and method may also comprise at least one reference electrode and one auxiliary electrode. In an embodiment, an applied potential protocol may be utilized and a resultant electrical potential may be measured between the at least one measurement, primary or secondary, electrode and at least one reference electrode. The systems and methods as described herein provide a technique for accurate measurement of pH in a range of sample types such as samples containing heavy metals or samples with low conductivity.

At 202, in an embodiment, a secondary electrode may be introduced into an aqueous sample. A secondary electrode may be referred to as a secondary measurement electrode or a secondary BDD electrode (see FIG. 1). In an embodiment, a secondary electrode may be used to measure a component or interferent of an aqueous sample. In an embodiment, the component or interferent may be a heavy metal. A heavy metal may be copper, manganese, or the like. In an embodiment, a secondary electrode may comprise either no pH sensitive carbon region or a pH sensitive carbon region smaller than the pH sensitive carbon region of a primary electrode. In an embodiment, a secondary electrode may have either no sp2 carbon region or a smaller sp2 carbon region as compared to a primary electrode. The secondary electrode may be of a BDD material which may comprise sp2 carbon material. In other words, a secondary electrode may be of a BDD material with an area of laser machined sp2 carbon area less than the sp2 carbon area of a primary electrode. In an embodiment, the sp2 carbon regions may be a plurality of laser machined areas. In an embodiment, the sp2 carbon may have a surface area. The sp2 carbon or carbon surface area may be a total area of all sp2 regions upon an electrode face.

In an embodiment, the measurement apparatus, or electrochemical apparatus, may be introduced into an aqueous sample. Alternatively, an aqueous sample may be introduced into a test chamber, for example, a test chamber of a measurement device. If the aqueous sample is introduced into the measurement device, the aqueous sample may be placed or introduced into a test chamber manually by a user or using a mechanical means, for example, gravity flow, a pump, pressure, fluid flow, or the like. For example, a water sample for pH testing may be introduced to a measurement or test chamber using a pump. In an embodiment, valves or the like may control the influx and efflux of the aqueous solution into or out of the one or more chambers, if present. Once the sample is introduced to the measurement system, the system may measure the pH of the sample, using steps as disclosed. In an embodiment, the measurement device may include one or more chambers in which the one or more method steps may be performed.

In an embodiment, the carbon region may be of a BDD material, BDD sp2 material, quinone structures, quinone-like structures, oxidized carbon structures, or the like. The carbon material may be laser machined upon the measurement electrode. The laser machining may be a plurality of laser machined areas or a continuous region. The laser machined region may be circular or circular-like in shape such as a circle, oval, or the like. In other words, other shapes of carbon regions are disclosed and may be laser machined to a particular use or configuration of a measurement electrode.

In an embodiment, the size, depth, or surface area of the laser machined sp2 region may be constructed for a particular use. In other words, the sp2 region may be machined in accordance with actual or expected pH ranges, heavy metal type, heavy metal concentration, or the like within a sample to be measured. In an embodiment, the sp2 carbon regions may be of a different number and/or diameter. For example, the diameter of a single sp2 laser machined area may be larger or smaller. As another example, the number of sp2 regions may be increased or decreased on a given electrode. The diameter and number of sp2 regions may be increased or decreased for primary and/or secondary electrodes.

In an embodiment, the electrodes may be fully or at least partially disposed in the volume of aqueous solution. For example, if the aqueous solution is introduced into a chamber having one or more electrodes, the aqueous solution may at least partially cover the one or more electrodes. As another example, the one or more electrodes may be partially disposed within the chamber with the other portion of the electrode outside the chamber. Thus, when the aqueous solution is introduced into the chamber it only covers the portion of the electrodes that are within the chamber.

At 203, the system and method may identify a characteristic of the aqueous sample. In an embodiment, the characteristic may be a pH or measurement of a component or interferent in the aqueous sample. For example, a reference, an auxiliary, and a primary electrode may measure a pH of an aqueous sample. For example, a reference, an auxiliary, and a secondary electrode may measure an electrochemical component of an interferent or a heavy metal. In an embodiment, there may be more than two measurement electrodes for measurement. Different measurement electrodes may measure components or characteristics sequentially, simultaneously, at periodic intervals, or the like. Different measurement electrode may use a common reference and a common auxiliary electrode.

The system may measure an electrical potential of the volume of aqueous sample across at least one measurement electrode and at least one reference electrode. The system and method may have either, one measurement electrode and one reference electrode, or have a plurality of measurement electrodes and a plurality of reference electrodes.

The electrical potential may be measured across one or more electrodes, for example, a series of electrodes. In an embodiment, the primary measurement electrode may be used to measure the electrical potential attributable to a pH of an aqueous sample. The measurement electrode may contain a carbon region. The carbon region is described above. The carbon region may be laser machined. In an embodiment, the carbon region may be a BDD, BDD sp2, sp2 carbon, oxidized carbon structure, quinone, quinone-like structure or the like. The carbon region may be a continuous region in a circular-like shape.

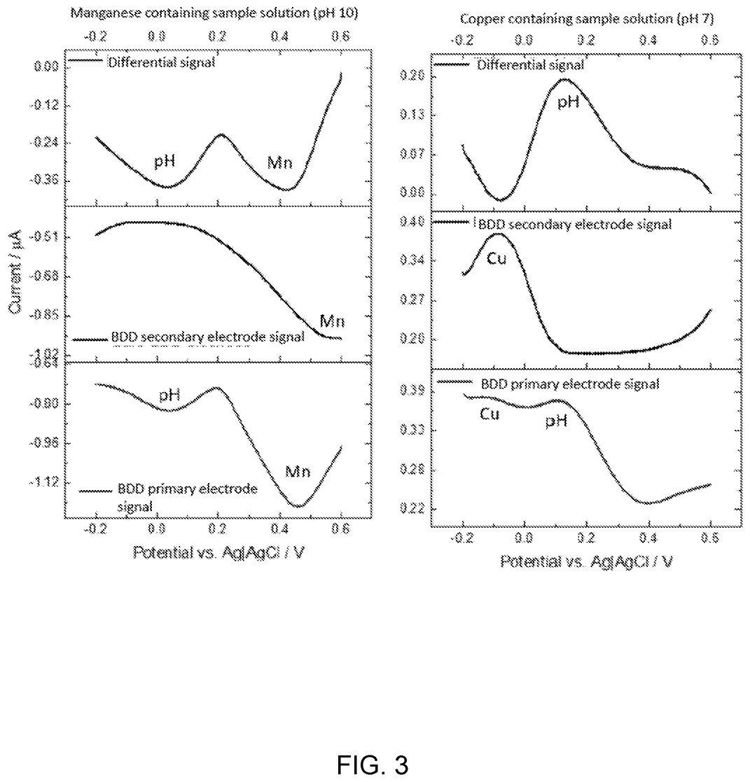

Referring to FIG. 3, an example measurement of the system and method is illustrated. Two example heavy metal components are illustrated. Other components and or heavy metals may be measured and are disclosed. In an embodiment, a primary electrode may measure a pH component from a primary electrode described above. In an embodiment, a secondary electrode may measure the component of a heavy metal, such as manganese or copper, from a secondary electrode. In an embodiment, an accurate determination of a pH value may be determined. For example, a current—voltage measurement from a secondary electrode may be subtracted from a current—voltage measurement from a primary electrode to give a differential signal. In other words, as an example, a measured heavy metal component may be subtracted from a measured pH component. In an embodiment, a measurement from a primary and secondary electrode may be express as a ratio.

Referring back to FIG. 2. at 204, if the system cannot identify a pH or characteristic of the aqueous sample, the system may continue to measure electrical responses from the electrodes of the system at 201 and/or 202. Additionally or alternatively, the system may trigger an alarm, shut down, alter flow control of the aqueous sample, or the like. However, if, at 205, a pH of the aqueous sample may be determined, the system may output the pH of an aqueous solution. An output may be in the form of a display, storing the data to a memory device, sending the output through a connected or wireless system, printing the output, or the like. The system may be automated, meaning the system may automatically output the identified pH. The system may also have associated alarms, limits, or predetermined thresholds. For example, if a measured pH reaches a threshold, the system may trigger an alarm, adjust the pH of the aqueous solution, alter the flow of the aqueous solution, or the like. Data may be analyzed in real-time, stored for later use, or any combination thereof.

Circuitry may control the measurement (e.g, potential, pH, etc.) to one or more series of electrodes such that different electrical signals may be applied and/or measured with respect to the volume of aqueous sample. The circuitry represents an example embodiment for at least one measurement electrode with a first carbon region, and a pH sensitive carbon region with at least one reference electrode.

The various embodiments described herein thus represent a technical improvement to conventional methods and instrument for pH measurement. Using the techniques as described herein, an embodiment may use a method and device for an instrument for pH measurement. This is in contrast to conventional methods with limitations mentioned above. Such techniques provide a better method to construct and an instrument for pH measurement.

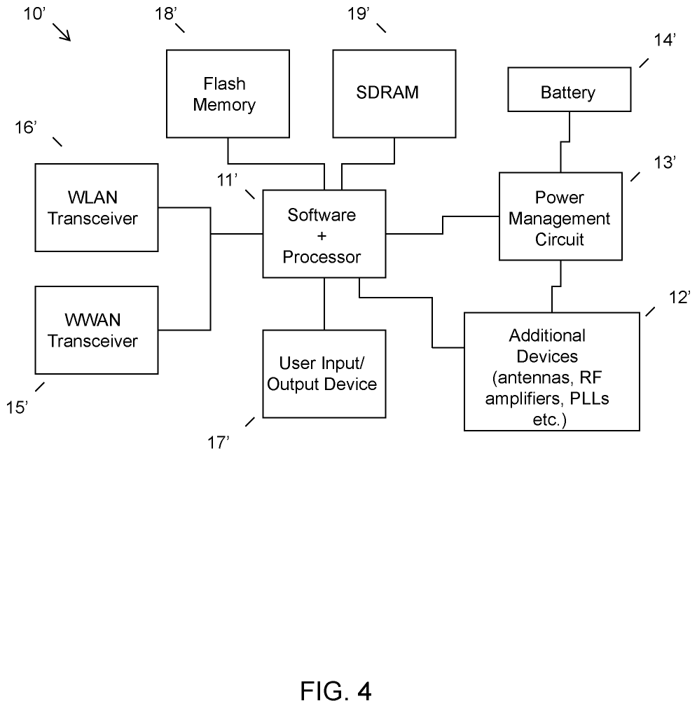

While various other circuits, circuitry or components may be utilized in information handling devices, with regard to an instrument for pH measurement according to any one of the various embodiments described herein, an example is illustrated in FIG. 4. Device circuitry 10′ may include a measurement system on a chip design found, for example, a particular computing platform (e.g., mobile computing, desktop computing, etc.) Software and processor(s) are combined in a single chip 11′. Processors comprise internal arithmetic units, registers, cache memory, busses, I/O ports, etc., as is well known in the art. Internal busses and the like depend on different vendors, but essentially all the peripheral devices (12′) may attach to a single chip 11′. The circuitry 10′ combines the processor, memory control, and I/O controller hub all into a single chip 11′. Also, systems 10′ of this type do not typically use SATA or PCI or LPC. Common interfaces, for example, include SDIO and I2C.

There are power management chip(s) 13′, e.g., a battery management unit, BMU, which manage power as supplied, for example, via a rechargeable battery 14′, which may be recharged by a connection to a power source (not shown). In at least one design, a single chip, such as 11′, is used to supply BIOS like functionality and DRAM memory.

System 10′ typically includes one or more of a WWAN transceiver 15′ and a WLAN transceiver 16′ for connecting to various networks, such as telecommunications networks and wireless Internet devices, e.g., access points. Additionally, devices 12′ are commonly included, e.g., a transmit and receive antenna, oscillators, PLLs, etc. System 10′ includes input/output devices 17′ for data input and display/rendering (e.g., a computing location located away from the single beam system that is easily accessible by a user). System 10′ also typically includes various memory devices, for example flash memory 18′ and SDRAM 19′.

It can be appreciated from the foregoing that electronic components of one or more systems or devices may include, but are not limited to, at least one processing unit, a memory, and a communication bus or communication means that couples various components including the memory to the processing unit(s). A system or device may include or have access to a variety of device readable media. System memory may include device readable storage media in the form of volatile and/or nonvolatile memory such as read only memory (ROM) and/or random access memory (RAM). By way of example, and not limitation, system memory may also include an operating system, application programs, other program modules, and program data. The disclosed system may be used in an embodiment of an instrument for pH measurement.

As will be appreciated by one skilled in the art, various aspects may be embodied as a system, method or device program product. Accordingly, aspects may take the form of an entirely hardware embodiment or an embodiment including software that may all generally be referred to herein as a “circuit,” “module” or “system.” Furthermore, aspects may take the form of a device program product embodied in one or more device readable medium(s) having device readable program code embodied therewith.

It should be noted that the various functions described herein may be implemented using instructions stored on a device readable storage medium such as a non-signal storage device, where the instructions are executed by a processor. In the context of this document, a storage device is not a signal and “non-transitory” includes all media except signal media.

Program code for carrying out operations may be written in any combination of one or more programming languages. The program code may execute entirely on a single device, partly on a single device, as a stand-alone software package, partly on single device and partly on another device, or entirely on the other device. In some cases, the devices may be connected through any type of connection or network, including a local area network (LAN) or a wide area network (WAN), or the connection may be made through other devices (for example, through the Internet using an Internet Service Provider), through wireless connections, e.g., near-field communication, or through a hard wire connection, such as over a USB connection.

Example embodiments are described herein with reference to the figures, which illustrate example methods, devices and products according to various example embodiments. It will be understood that the actions and functionality may be implemented at least in part by program instructions. These program instructions may be provided to a processor of a device, e.g., a measurement device such as illustrated in FIG. 1, or other programmable data processing device to produce a machine, such that the instructions, which execute via a processor of the device, implement the functions/acts specified.

It is noted that the values provided herein are to be construed to include equivalent values as indicated by use of the term “about.” The equivalent values will be evident to those having ordinary skill in the art, but at the least include values obtained by ordinary rounding of the last significant digit.

This disclosure has been presented for purposes of illustration and description but is not intended to be exhaustive or limiting. Many modifications and variations will be apparent to those of ordinary skill in the art. The example embodiments were chosen and described in order to explain principles and practical application, and to enable others of ordinary skill in the art to understand the disclosure for various embodiments with various modifications as are suited to the particular use contemplated.

Thus, although illustrative example embodiments have been described herein with reference to the accompanying figures, it is to be understood that this description is not limiting and that various other changes and modifications may be affected therein by one skilled in the art without departing from the scope or spirit of the disclosure.

What is claimed is:

1. A device for measuring pH in an aqueous sample, comprising:
a primary electrode comprising a boron doped diamond-based electrode and a primary carbon region comprising a pH sensitive sp2 carbon region, wherein the primary carbon region comprises a portion of the surface area of a face of the primary electrode;
a secondary electrode comprising a boron doped diamond-based electrode and a secondary carbon region, wherein the secondary carbon region comprises a portion of the surface area of a face of the secondary electrode, the portion of the surface area of a face of the secondary electrode being less than the portion of the surface area of a face of the primary electrode;
at least one reference electrode;
at least one auxiliary electrode; and
a memory storing instructions executable by a processor to identify a pH of an aqueous sample by measuring an electrical potential between the at least one reference electrode and at least one of: the primary electrode and the secondary electrode.

2. The device of claim 1, wherein the primary carbon region comprises a plurality of pH sensitive carbon regions.

3. The device of claim 1, wherein the portion of the surface area of a face of the secondary electrode is zero.

4. The device of claim 1, wherein each of the primary carbon region and the secondary carbon region is a laser micromachined area.

5. The device of claim 1, wherein each of the primary carbon region and the secondary carbon region comprises oxidized carbon structures.

6. The device of claim 5, wherein the oxidized carbon structures further comprise quinone-like groups.

7. The device of claim 1, wherein the measured electrical potential between the at least one reference electrode and the primary electrode corresponds to a pH value.

8. The device of claim 1, wherein the measured electrical potential between the at least one reference electrode and the secondary electrode corresponds to an electrochemical signal comprising an interference signal.

9. The device of claim 1, wherein the identification of the pH value is determined by subtracting a potential from the secondary electrode from a potential from the primary electrode.

10. The device of claim 1, wherein the aqueous sample comprises a heavy metal.

11. A method for measuring pH in an aqueous sample with a carbon region electrode, comprising:
introducing an aqueous sample into a measurement device, wherein the measurement device comprises:
a primary electrode comprising a boron doped diamond-based electrode and a primary carbon region with a primary carbon region surface area, wherein the primary carbon region comprises a pH sensitive sp2 carbon region;
a secondary electrode comprising a boron doped diamond-based electrode and a secondary carbon region with a secondary carbon region surface area less than the primary carbon region surface area;
at least one reference electrode;
at least one auxiliary electrode; and
identifying a pH of an aqueous sample by measuring an electrical potential between the at least one reference electrode and at least one of: the primary electrode and the secondary electrode.

12. The method of claim 11, wherein the primary carbon region comprises a plurality of pH sensitive carbon regions.

13. The method of claim 11, wherein the portion of the surface area of a face of the secondary electrode is zero.

14. The method of claim 11, wherein each of the primary carbon region and the secondary carbon region is a laser micromachined area.

15. The method of claim 11, wherein each of the primary carbon region and the secondary carbon region comprises oxidized carbon structures.

16. The method of claim 15, wherein the oxidized carbon structures further comprise quinone-like groups.

17. The method of claim 11, wherein the measured electrical potential between the at least one reference electrode and the primary electrode corresponds to a pH value.

18. The method of claim 11, wherein the measured electrical potential between the at least one reference electrode and the secondary electrode corresponds to an electrochemical signal comprising an interference signal.

19. The method of claim 11, wherein the identification of the pH value is determined by subtracting a potential from the secondary electrode from a potential from the primary electrode.

20. A system for measuring pH in an aqueous sample, comprising:
a storage device having code stored therewith, the code being executable by the processor and comprising:
code that introduces an aqueous sample into a measurement device, wherein the measurement device comprises:
a primary electrode comprising a boron doped diamond-based electrode and a primary carbon region with a primary carbon region surface area, wherein the primary carbon region comprises a pH sensitive sp2 carbon region;
a secondary electrode comprising a boron doped diamond-based electrode and a secondary carbon region with a secondary carbon region surface area less than the primary carbon region surface area;
at least one reference electrode;
at least one auxiliary electrode; and
code that identifies a pH of an aqueous sample by measuring an electrical potential between the at least one reference electrode and at least one of: the primary electrode and the secondary electrode.
